(19) United States
(12) Patent Application Publication
(10) Pub. No.: US 2006/0120993 A1
(43) Pub. Date: Jun. 8, 2006

(54) Treatment of lesions of the soft tissues

(75) Inventors: Alexander D. Vilesov, St. Petersburg (RU); Marina S. Vilesova, St. Petersburg (RU); Viktor N. Balin, St. Petersburg (RU); Natalia I. Eisenstadt, St. Petersburg (RU); Vasily V. Mikhailov, St. Petersburg (RU)

Correspondence Address: HOUSTON ELISEEVA, 4 MILITIA DRIVE, SUITE 4, LEXINGTON, MA, 02421, US

(73) Assignee: OOO Delsi, St. Petersburg (RU)

(21) Appl. No.: 11/337,433

(22) Filed: Jan. 23, 2006

Related U.S. Application Data
(63) Continuation of application No. PCT/IB04/02358, filed on Jul. 22, 2004.

(30) Foreign Application Priority Data
Jul. 24, 2003 (RU) 2003123513

(51) Int. Cl. A61K 31/785; A61K 38/39; A61K 31/717; A61K 33/14

(52) U.S. Cl. 424/78.27; 424/78.28; 514/2; 514/54; 514/57; 424/680

(57) ABSTRACT

The invented remedy allows cleaning up the area from necrotic tissues and facilitating reduced perifocal oedema and infiltration as well as lessening pain syndrome during suppurative inflammation diseases. The remedy can be applied both in the hospital and field settings.

### RELATED APPLICATIONS

This application is a Continuation of PCT application serial number PCT/IB2004/002358 filed on Jul. 22, 2004 which in turn claims priority from Russian application serial number 2003123513 filed on Jul. 24, 2003 both of which are incorporated herein by reference in their entirety.

### FIELD OF THE INVENTION

An invention is related to the field of medicine, particularly, to surgery, and can be used for treatment of the suppurative inflammation lesions of the soft tissues.

### BACKGROUND OF THE INVENTION

Known in the art is a wound coating and method for its preparation (RU No 2091082, A61L 15/30, 1997). The coating is a multi-layer film made of the fluorinated rubber and water-soluble polysaccharides of the plant origin, combined in certain ratio. The coating is obtained by mixing of the fluorinated rubber latex and water solution of the polysaccharide to homogeneity, and then spreading out the resulting mass on the flat surface and drying.

Known in the art is a remedy for wound healing (RU No 2115436, A61L 15/24, 1995). The remedy is made of the co-polymer of the vinyl alcohol, vinyl acetate and vinyl glutarate as a dry powder with the particle size 10-1500 μM also containing a drug selected from the following group: anitimicrobial, proteolytic enzymes or local anesthetic.

Known in the art is a method for healing of the infected wounds (RU application No 94033866/14, A61K 39/106, 1994). The method consists in a surgical and mechanical treatment of the wound, then drainage and washing, and carrying out a restoration surgery and local drug therapy. Washing is accomplished by a single daily treatment with a pilastin solution, 0.25-0.8 dose/mL over 3-5 days, whereas a local therapy is conducted by applying a bandage soaked in the water solution of pilastin of the same concentration for a period of 24-48 h, over 5-8 days.

It is known that for healing of the local festering wounds during the exudative phase, the compounds with hyper-osmolar properties can be used in order to facilitate an outflow of the wound discharge from the wound to the bandage. (Wounds and Wound Infections. Manual for Doctors. Eds. M. I. Kuzin, B. M. Kostyuchenok, Moscow, Medicine, 1990, p. 281). Among the hypertonic solutions that received widespread distribution, a 10% NaCl solution is known. The method has initially been scientifically justified in 1890 by M. Ya. Preobrazhensky and studied both experimentally and clinically in the W. E. Wright's clinic. (Struchkov, V. I.; Grigoryan, A. V.; Gostishchev, V. K., Festering Wound, Moscow, Medicine, 1975, pp. 166-168).

Among the drawbacks of the above-mentioned remedies is a short-term therapeutic effect due to dilution with wound exudates and quick drying (2-3 h), which requires frequent change of the bandages (not less than 3 times a day).

Known in the art is a method for healing of the festering wounds using granulated adsorbents based on the chemically interwoven polymers, for example, gelevin. (Theory and Practice of the Local Healing of the Festering Wounds, Ed. B. M. Datsenko, Kiev, Health, 1995, pp. 175-177).

The main disadvantage is low or moderate osmotic properties of the adsorbents and also the fact that the most of the adsorption occurs over the first few minutes of the contact and then decreases significantly that requires repeated daily changes of the bandage.

### SUMMARY OF THE INVENTION

The tasks which solutions the inventions are targeting consist in reducing timeframes of the main stages of the wound healing process and decreasing duration of the inpatient treatment for the patients with suppurative inflammation lesions of the soft tissues by virtue of prolonged maintenance of the required drug concentrations in the wound.

The formulated problems have been addressed in the following manner.

The remedy for healing of the suppurative inflammation lesions of the soft tissues contains a polymer and has an appearance of the micro-granules which matrix is composed of at least one cross-linked polymer selected from the group containing sodium alginate, gelatin, pectin, carraginan, agar-agar, sodium salt of carboxymethylcellulose, copolymer of the acrylic acid and butyl acrylate, and their mixture, and additives, that are hyper-osmolar, antiseptic, anesthetic, and, if necessary, antioxidants, in the following ratios, weight %:

Matrix:

| | |
|---|---|
| Indicated cross-linked polymer | 11.0-29.0, |
| Additive: | |
| hyper-osmolar | 70.0-80.0, |
| antiseptic | 0.5-5.5, |
| anesthetic | 0.5-2.5, |
| antioxidant | 0.05-1.0, |

while the granules that have the size of 500-3000 μM are shaped spherically or close to the spherical form.

As hyper-osmolar compounds, the remedy contains sodium chloride (NaCl) or magnesium chloride (MgCl₂), or the sea salt. As antiseptic compound, the remedy contains lidocaine or nitazole. As anesthetic compound, the remedy contains lidocaine, or trimecaine, or pyromecaine hydrochloride. As antioxidant, the remedy contains olifen, or carnasine, or emoxipine.

The method of treatment of the suppurative inflammation lesions of the soft tissues using the above-mentioned remedy along with antibacterial, anti-inflammatory, desensitizing and disintoxication therapy that is different by placing the micro-granules via post-surgical or fresh wound into the suppurative inflammation lesion and filling not more than half the volume of the wound cavity followed by the wound drainage and applying an aseptic bandage that is replaced along with the granules once a day over 2-3 days.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### DETAILED DESCRIPTION OF THE PREFERRED EMBODIMENTS

The proposed remedy MIKPOL has an appearance of loose powder consisting of micro-granules. The micro-granules are composed of the polymer matrix and functional additives, that are primarily salts creating a local hyper-osmolar effect in the suppurative inflammation lesion, and also anesthetic and antiseptic compounds, both in combination, and separately. The granules are obtained by granulation of the proposed compositions followed by drying and sterilization. The application of the above-mentioned components in granulated form rather than in native state provides a general effect of the prolonged action of the encapsulated active components, salts, first of all. Moreover, one can control a rate of the release of the components (salts) from the granule to the wound by changing the ratio of the salt and polymer matrix, and also by altering a degree of cross-linking of the polymer matrix that is supported by the studies of the release kinetics followed by change of the salt concentration in the external part of the solution in vitro. The use of anesthetic compounds as a component of the micro-granules reduces pain threshhold and even relieve pain syndrome.

MIKPOL is introduced via surgical opening or fresh wound directly into the suppurative inflammation nidus or wound. In the process of the soaking of the micro-granules situated in the wound by the exudates, a mixture of the micro-granule components gets diffused into the lesion thus maintaining an increased osmotic pressure as compared to the interstitial liquid and blood plasma. This provides a centripetal flow of the tissue liquid from the perifocal area directly to the suppurative cavity and further down to the bandage via drainage, facilitating an accelerated excretion of the excess of fluids and metabolites from the inflamed tissues and swollen adjacent tissues and reducing oedema. Therefore, it improves the conditions for restoration of the microcirculation in the area of inflammation, the inflow of the fresh components of the humoral and cell system of immuno-protection is stimulated, and also the concentration of the drugs delivered perenteral and supplied from the vasculature is increased. The activity of the micro-granules in time is regulated through cross-polymerization in the preformed granules that affords possessing a stable diffusion of the microelements over the period of time not less than 24 h. Moreover, the micro-granules have an ability to absorb a significant amount of the exo- and endo-toxins from the wound exudates and also to assert a hemo-static, anesthetic, and antiseptic effects.

The conducted study of physico-chemical and healing properties led us to conclude that by combining polymer micro-granules with different content of additives and different cross-polymerization pattern, one can regulate the activity of the hyper-osmolar component in time, that allows to provide a steady diffusion rate to the environment over the period of time that is not less then 24 h. By possessing the stable chemical bonds between polymer chains, the granules are capable retaining a stable shape, which given their low reciprocal adhesion permits a non-obstructed inflow of the released exudates to the bandage via inter-granular space. This distinguishes it favorably from the known analogs which form continues gel layer on the surface of the wound, and thus manifests a critical advantage for the suppurative inflammation that is typically accompanied by prominent exudation. The treatment with MIKPOL is conducted in the first phase of the inflammation process till complete termination of the pronounced exudation events in the festering wound.

The method of treatment with MIKPOL is conducted along with general line of antibacterial, anti-inflammation, desensitizing and disintoxication therapy. Microgranules are introduced into fresh wound or suppurative necrotic nidus so that about half of the volume of the wound or suppurative nidus is filled. The cavity is then drainaged by any know method, and aseptic bandage is applied. The bandage and granules are replaced once a day over next 2-4 days. In the cases with particularly abundant exudation, which is determined by soaking of the bandage, the bandage could be replaced twice a day. The breadth of therapeutic applications may be extended by complimentary incorporation into the granules of any drug substances. The remedy can be used both in clinical and field setting. Of particular importance, is possible application in the emergency situation and for terrorist attacks victims in the medical units of the Ministry of Defense and Ministry of the Extreme Situations, and also by the Ministry of Health for inpatient and outpatient treatment of the suppurative inflammation complications of the trauma of different localization. MIKPOL allows qualitatively improve treatment of the patients, decrease the number of suppurative septic complications in the cases of mechanical and gun-inflicted trauma, shorten the rehabilitation time, and also reduce the labor allocation for the medical personnel involved in patient care in the surgical departments of the Ministry of Health and Ministry of the Extreme Situations.

The proposed inventions are illustrated by examples. The Table lists the compositions of MIKPOL that were used for treatment of the patients with suppurative inflammation lesions.

| | TABLE | | | | |
|---|---|---|---|---|---|
| | Sample, % weight | | | | |
| Components | No 1 | No 2 | No 3 | No 4 | No 5 |

Hyper-osmolar compounds:

| | | | | | |
|---|---|---|---|---|---|
| Sodium chloride | | 79.0 | | | |
| Magnesium chloride | 73.0 | | | | |
| Sea salt | | | 79.0 | 80.0 | 81.0 |

Antibacterial compounds

| | | | | | |
|---|---|---|---|---|---|
| Dioxidine | | 1.0 | | 0.5 | |
| Nitazole | 0.5 | | 0.5 | | 1.0 |

Anesthetic compounds:

| | | | | | |
|---|---|---|---|---|---|
| Lidocaine | | | 1.0 | | 1.0 |
| hydrochloride | | | | | |
| Trimecaine | | | | 0.5 | |
| hydrochloride | | | | | |
| Pyromecaine | 0.5 | 0.5 | | | |
| hydrochloride | | | | | |

Antioxidant compounds:

| | | | |
|---|---|---|---|
| Carnasine | 0.5 | | |
| Olifen | | 0.5 | |
| Emoxipine | | | 1.0 |

Polymers:

| | | | | | |
|---|---|---|---|---|---|
| Sodium alginate | 26.0 | | | | 16.0 |
| Sodium alginate + | | 19.0 | | | |
| gelatin | | | | | |
| Pectin | | | | 19.0 | |
| Sodium alginate + | | | 19.0 | | |
| copolymer of the | | | | | |
| acrylic acid and | | | | | |
| butylacrylate | | | | | |

### EXAMPLE 1

The patient N., 34 years old, was hospitalized with an acute odontogenic localized osteomyelitis of the lower jaw adjacent to the 36^{th} tooth, complicated by the flegmon of the mouth floor, in overall fair conditions. After lancing and drainage on the day of hospitalization under general anesthesia of the left submaxillary and lower chin areas, MIKPOL No. 1 was introduced into the suppurative necrotic nidus to fill half the volume of the cavity. A post-surgical wound was drainaged with two tubular double gap drainages. The bandaging that was done twice a day and the fractional wound wash was also completed via drainages using 0.02% chlorohexedine. The swollen granules that resided in the wound for a day were washed out unobstructed whereas a new portion of the MIKPOL of the same volume was introduced to the wound. An anti-bacterial, anti-inflammatory, desensitizing and disintoxication therapy were carried out. As a result, by the beginning of the second day, the body temperature normalized. The pus discharge terminated on the day 3, the wound cleaned from necrosis areas that permitted to remove tubular drainage and make a transfer to local application of the wound-healing remedies (activin-gel). By the day 4, the visible nidi of granulation tissues appeared. Planimetrically, the volume of the post-surgical wound reduced from 12.4 cm³ to 5.6 cm³ by the day 6. On the day 6, the secondary stitches were applied. The period of the inpatient treatment totaled 8 days.

### EXAMPLE 2

Patient C., 33 years old, admitted with acute adenoflegmon of the right submaxillary area. Upon admission, a lancing and drainage of the adenoflegmon were conducted. Local treatment was carried out using MIKPOL No. 2 that was administered to the post-surgical wound once a day to fill approximately half the volume of the suppurative cavity. Besides that, a general anti-bacterial and anti-inflammatory treatment was carried out. By the second day, a termination of the exudates processes and cleaning of the cavity from necrotic tissues were registered. The cytology study recorded a transition to the regenerative stage by the day 4 of the treatment, and then the traditional local wound healing remedies were commenced. The double reduction of the wound size to 5.99 cm³ as determined by the planimetrical study, occurred by the day 5 of treatment. The secondary stitches were applied on the day 6 of the treatment.

### EXAMPLE 3

The patient Sh., 41 years old, was treated for an acute odontogenic localized osteomyelitis of the lower jaw, complicated by the flegmon of the left submaxillary area. After a surgery with lancing and drainage, the local treatment was conducted with MIKPOL No. 3. During bandaging that was conducted once a day, the cavity was washed out with antiseptics via tubular drainage and the micro-granules were introduced to fill half the volume. By the next bandaging, the granules became swollen by absorbing the wound discharge. They were easily removed from the wound during bandaging when washing the wound with antiseptic via tubular drainage, after that a new portion of the MIKPOL was applied. Moreover, general anti-bacterial and anti-inflammatory treatments were conducted. As a result of the treatment, the exudates events ceased on the day 2 that allowed removing the tubular drainage on the day 3 and making a transition to the wound-healing ointment bandages. A cytology study determined a regenerative type of the cytogram on the day 4. A planimetrically measured reduction of the size of the wound occurred on the day 5. The secondary stitches were applied on the day 6 after beginning of the treatment.

### EXAMPLE 4

Patient Z, 30 years old. Admitted with a double-sided non-consolidate fracture of the lower jaw in the area of 36^{th} and 44th teeth complicated by the flegmon of the left submaxillary area. The lancing and drainage surgery were performed; the lower jaw was immobilized using Vasiliev's splints with inter-jaw rubber tug. The local treatment of the post-surgical wound was done using MIKPOL No. 4, by filling half the volume of the suppurative cavity and applying an aseptic bandage. A bandage replacement frequency was once a day. As a result of the treatment, the exudative processes were suppressed by the day 3, whereas an infiltration of the soft tissues disappeared by the day 5. Beginning from the day 4 since onset of the treatment, a local application of the healing drugs commenced (actovegin-gel). The transition of cytology picture from degenerative inflammatory to regenerative was observed on the day 5. The volume of the wound was 10.3 cm³ on the day 2, and 4.8 cm³ on the day 6. A post-surgical wound healed by secondary stretching by the day 12 from beginning of the treatment. A consolidation of the fracture of the lower jaw proceeded without complications.

### EXAMPLE 5

The patient L, 30 years old, was treated for the acute odontogenic osteomielitis of the lower jaw adjacent to the 48^{th} tooth, complicated by the phlegmon of the moth floor. An excision surgery and drainage was conducted at the left and right submaxillary, sublingual and lower chin areas. During surgery the patient had an abundant malodorous gray pus discharge. The soft tissues of the moth floor had necrotic alterations; there were indications of myocitis and fasciitis. With a background of general therapy, MIKPOL No. 5 was applied locally by filling necrotic cavities during bandaging that were performed twice a day after wound dialysis using antiseptics. 24 h after surgery, there was no detected malodor from the wound and the amount of the wound discharge decreased significantly. The volume of the cavity was determined planimetrically to be 41.6 cm³. Already after 3 days, the necrotic processes went into a recession, the wound has cleaned, and exudation processes halted. The tubular drainage was removed and the ointment bandages began to be applied locally. A cytology picture by the day 5 commenced to display the traits of regenerative process manifested by the macroscopic granulation. By the day 6, the volume of the cavities was 23.5 cm³. The early secondary stitches bringing together edges of the wound were applied. The patient was released for the outpatient treatment on the day 10 after the surgery.

The invented remedy allows cleaning up the area from necrotic tissues and facilitating reduced perifocal oedema and infiltration as well as lessening pain syndrome during suppurative inflammation diseases. The remedy can be applied both in the hospital and field settings.

While this invention has been particularly shown and described with references to preferred embodiments thereof, it will be understood by those skilled in the art that various changes in form and details may be made therein without departing from the scope of the invention encompassed by the appended claims.

What is claimed is:

1. A composition for treating soft tissue suppurative inflammation lesions, including wounds, flegmons or combinations thereof, wherein the composition comprises granules that include:
a. a crossed-linked polymer selected from the group consisting of an alginate, gelatin, pectin, carrageenan, agar, a cellulose derivative, a copolymer of acrylic acid and an acrylate, and any combination thereof; and
b. a functional additive component including a hyperosmolar compound.

2. The composition of claim 1, wherein the hyperosmolar compound is selected from the group consisting of sodium chloride, magnesium chloride, sea salt, and any combination thereof.

3. The composition of claim 1, wherein the crossed-linked polymer is present in the composition in an amount in the range of from about 11 to about 29 weight percent and the hyperosmolar compound is present in the composition in an amount in the range of from about 70 to about 80 weight percent.

4. The composition of claim 1, wherein the functional additive component further includes a compound selected from the group consisting of an antiseptic agent, an anesthetic agent, an antioxidant, and any combination thereof.

5. A method for treating soft tissue suppurative inflammation lesions, including wounds, flegmons or combinations thereof, wherein the method comprises administering to a soft tissue suppurative lesion granules that include:
a. a crossed-linked polymer selected from the group consisting of alginate, gelatin, pectin, carrageenan, agar, cellulose derivative, copolymer of acrylic acid and an acrylate, and any combination thereof; and
a. a functional additive component including a hyperosmolar compound.

6. The method of claim 5, wherein the granules have a size in the rage of from about 500 to about 3000 microns.

7. The method of claim 5, wherein the granules are spherical or approximately spherical.

8. The method of claim 5, wherein the hyperosmolar compound is selected from the group consisting of sodium chloride, magnesium chloride, sea salt, and any combination thereof.

9. The method of claim 5, wherein the crossed-linked polymer is present in the granules in an amount in the range of from about 11 to about 29 weight percent and the hyperosmolar compound is present in the granules in an amount in the range of from about 70 to about 80 weight percent.

10. The method of claim 5, wherein the functional additive component further includes a compound selected from the group consisting of an antiseptic agent, an anesthetic agent, an antioxidant, and any combination thereof.

11. The method of claim 5, further comprising administering at least one therapy selected from the group consisting of antibacterial therapy, anti-inflammatory therapy, desensitizing therapy, disintoxication therapy, and any combination thereof.

12. The method of claim 11, wherein the at least one therapy is administered locally.

13. The method of claim 11, wherein the at least one therapy is administered systemically.

14. The method of claim 5, wherein the granules are administered by introducing the granules into a post surgical wound, a fresh wound or a suppurative necrotic nidus.

15. The method of claim 14, wherein not more than half the volume of the wound cavity or of the suppurative necrotic nidus is filled with granules.

16. The method of claim 5, further comprising draining exudate from the suppurative inflammation lesion.

17. The method of claim 5, wherein the release rate of the functional additive component from the granules is controlled.

18. Granules for local therapy, the granules comprising:
a. a crossed-linked polymer selected from the group consisting of an alginate, gelatin, pectin, carrageenan, agar, a cellulose derivative, a copolymer of acrylic acid and an acrylate, and any combination thereof; and
b a functional additive component including a hyperosmolar compound.

19. The granules of claim 18, wherein the granules have a size in the rage of from about 500 to about 3000 microns.

20. The granules of claim 18, wherein the granules are spherical or approximately spherical.

21. The granules of claim 18, wherein the hyperosmolar compound is selected from the group consisting of sodium chloride, magnesium chloride, sea salt and any combination thereof.

22. The granules of claim 18, wherein the crossed-linked polymer is present in the granules in an amount in the range of from about 11 to about 29 weight percent and the hyperosmolar compound is present in the granules in an amount in the range of from about 70 to about 80 weight percent.

23. The granules of claim 18, wherein the functional additive component further includes a compound selected from the group consisting of an antiseptic agent, an anesthetic agent, an antioxidant, and any combination thereof.

24. A method for treating a flegmon, the method comprising:
a. lancing the flegmon; and
b. administering at the lanced site granules that include:
i. a crossed-linked polymer selected from the group consisting of alginate, gelatin, pectin, carrageenan, agar, cellulose derivative, copolymer of acrylic acid and an acrylate, and any combination thereof; and
ii. a functional additive component including a hyperosmolar compound.
